(19) United States
(12) Patent Application Publication
(10) Pub. No.: US 2022/0087601 A1
(43) Pub. Date: Mar. 24, 2022

(54) ELECTRONIC DEVICE OF DETECTING APNEA, AND COMPUTER-READABLE STORAGE MEDIUM

(71) Applicant: BOE TECHNOLOGY GROUP CO., LTD., Beijing (CN)

(72) Inventors: Bao Fu, Beijing (CN); Hui Du, Beijing (CN); Guifang Meng, Beijing (CN); Yajuan Wang, Beijing (CN)

(21) Appl. No.: 17/446,364

(22) Filed: Aug. 30, 2021

(30) Foreign Application Priority Data
Sep. 23, 2020 (CN) 202011012215.7

(51) Int. Cl. A61B 5/00; A61B 5/369; A61B 5/021; A61B 5/145; A61B 5/08

(52) U.S. Cl. CPC A61B 5/4818; A61B 5/369; A61B 5/4812; A61B 5/08; A61B 5/6803; A61B 5/6821; A61B 5/14542; A61B 5/02116

(57) ABSTRACT

There is provided an electronic device for detecting apnea and a computer-readable storage medium. The electronic device includes: at least one of an electroencephalographic sensor configured to detect electroencephalographic data of a user or a nasal airflow sensor configured to detect nasal airflow data of the user; and a controller communicatively coupled to the at least one of the electroencephalographic sensor or the nasal airflow sensor. The controller is configured to determine a candidate apnea event based on at least the electroencephalographic data or the nasal airflow data.

### CROSS-REFERENCE TO RELATED APPLICATION

This application claims the priority of Chinese Patent Application No. 202011012215.7 filed on Sep. 23, 2020, the content of which is incorporated herein by reference.

### TECHNICAL FIELD

The present disclosure relates to a field of medical treatment and health, and in particular to an electronic device of detecting apnea and a computer-readable storage medium.

### BACKGROUND

As one of common diseases of modern people, sleep apnea syndrome (SAS) is a sleep disorder in which breathing stops during sleep. A most common cause is an obstruction of a respiratory tract, which often ends with loud snoring, body twitching or arm swinging. Sleep apnea is accompanied by sleep defects, daytime naps, fatigue, and bradycardia or arrhythmia and electroencephalographic awakening.

SAS may be roughly classified into three types, including: (1) Obstructive Sleep Apnea (OSA), in which a relaxation of a soft tissue (for example, a root of a tongue) near a throat causes the obstruction of the upper respiratory tract, and narrowing of the respiratory tract leads to the sleep apnea; (2) Central Sleep Apnea (CSA), in which a respiratory central nervous system has been damaged by stroke and trauma and is unable to normally convey breathing instructions so that sleep respiratory function disorders; and (3) Mixed Sleep Apnea (MSA), which is a mixture of the obstructive sleep apnea and the central sleep apnea.

Since this disease always occurs during sleep, a patient is usually not clearly aware of an illness and is usually informed by someone other than the patient. Even if the patient suspect an existence of apnea, a relevant monitoring scheme of detecting the apnea generally requires the patient to wear a plurality of complex devices (e.g., electroencephalograph, electrocardiogram, actigraphy, oronasal airflow meter, etc.) in a designated sleep room of a hospital. On the one hand, the cost is high, and on the other hand, it has a negative impact on user's sleep and affects a monitoring result.

With a continuous enhancement of social development, people's life pressure is also increasing, and more and more people have sleep problems. Sleep apnea syndrome is an important problem in sleep. If not handled in time, the sleep apnea syndrome may cause other health hazards, such as obesity, cardiovascular disease and so on.

Therefore, there is a need to provide a convenient and practical solution of detecting and classifying the sleep apnea syndrome at home, so as to help the user achieve sleep monitoring at home and help determine a sleep apnea problem.

### SUMMARY

In order to at least partially solve or mitigate the above problems, a wearable electronic device of detecting apnea and a method of detecting apnea according to embodiments of the present disclosure are provided.

According to a first aspect of the present disclosure, there is provided an electronic device of detecting apnea, including: at least one of an electroencephalographic sensor configured to detect electroencephalographic data of a user or a nasal airflow sensor configured to detect nasal airflow data of the user; and a controller communicatively coupled to the at least one of the electroencephalographic sensor or the nasal airflow sensor, wherein the controller is configured to determine a candidate apnea event based on at least the electroencephalographic data or the nasal airflow data.

In some embodiments, the electronic device further includes: a photoelectric sensor communicatively coupled to the controller and configured to detect blood oxygen data of the user, wherein the controller is further configured to verify whether the candidate apnea event is an apnea event or not based on at least the blood oxygen data.

In some embodiments, the photoelectric sensor is further configured to detect pulse wave data of the user; and the controller is further configured to determine a type of the apnea event based on at least the pulse wave data in response to determining that the candidate apnea event is the apnea event.

In some embodiments, the controller is configured to determine the candidate apnea event based on at least the electroencephalographic data by: determining an occurrence of the candidate apnea event in response to the electroencephalographic data satisfying

$$\frac{σ + θ}{α + β} > A$$

where σ, θ, α and β represent an electroencephalographic signal level in a frequency band of 1 Hz to 4 Hz, an electroencephalographic signal level in a frequency band of 4 Hz to 8 Hz, an electroencephalographic signal level in a frequency band of 8 Hz to 13 Hz and an electroencephalographic signal level in a frequency band of 13 Hz to 30 Hz in the electroencephalographic data, respectively, and A represents a preset threshold. In some embodiments, σ, θ, α and β are obtained by performing a spectrum analysis on the electroencephalographic data acquired by the electroencephalographic sensor. In some embodiments, the controller is configured to determine the candidate apnea event based on at least the nasal airflow data by: determining an occurrence of the candidate apnea event in response to the nasal airflow data indicating that an airflow per unit time of the nasal airflow decreases by more than 50%. In some embodiments, the controller is further configured to determine a sleep stage of the user according to the electroencephalographic data. In some embodiments, the controller is configured to verify whether the candidate apnea event is an apnea event or not based on at least the blood oxygen data by: determining that the candidate apnea event is the apnea event in response to at least one of: a value of the blood oxygen data decreasing by greater than or equal to 5%, the value of the blood oxygen data decreasing by 4% to 5% with a duration of greater than 30 seconds, or the value of the blood oxygen data decreasing by 3% to 4% with a duration of greater than 30 seconds and with awakening.

In some embodiments, the controller is configured to determine a type of the apnea event based on at least the pulse wave data in response to determining that the candidate apnea event is the apnea event by: determining a first average pulse wave amplitude during the apnea event and a second average pulse wave amplitude prior to the apnea event based on the pulse wave data; and determining the type of the apnea event based on at least the first average pulse wave amplitude and the second average pulse wave amplitude. In some embodiments, the controller is configured to determine the type of the apnea event based on at least the first average pulse wave amplitude and the second average pulse wave amplitude by: calculating a ratio of the first average pulse wave amplitude to the second average pulse wave amplitude; determining the apnea event as a central apnea event in response to determining that the ratio is less than 0.9; and determining the apnea event as an obstructive apnea event in response to determining that the ratio is within a preset interval [1−ε, 1+ε], where 0<ε≤0.1. In some embodiments, the first average pulse wave amplitude and the second average pulse wave amplitude are determined by identifying characteristic points of the pulse wave data acquired by the photoelectric sensor. In some embodiments, the controller is further configured to determine a pulse rate of the user according to the pulse wave data. In some embodiments, the controller is configured to determine the type of the apnea event based on at least the first average pulse wave amplitude and the second average pulse wave amplitude by: determining the type of the apnea event based on at least the first average pulse wave amplitude, the second average pulse wave amplitude and the pulse rate.

In some embodiments, the wearable electronic device is an eye mask electronic device, the electroencephalographic sensor includes an electrode arranged on the eye mask electronic device close to a forehead of the user, and the photoelectric sensor includes a photoelectric device arranged on the eye mask electronic device close to the forehead of the user. In some embodiments, the wearable electronic device further includes: a communication module configured to provide data associated with the apnea event and/or the type of the apnea event to outside in a wired or wireless manner.

According to a second aspect of the present disclosure, there is provided a method of detecting apnea, including: acquiring at least one of electroencephalographic data and nasal airflow data; and determining a candidate apnea event based on at least the electroencephalographic data or the nasal airflow data.

In some embodiments, the method further includes: acquiring blood oxygen data; and verifying whether the candidate apnea event is an apnea event or not based on at least the blood oxygen data.

In some embodiments, the method further includes: acquiring pulse wave data; and determining a type of the apnea event based on at least the pulse wave data in response to determining that the candidate apnea event is the apnea event.

In some embodiments, the determining the candidate apnea event based on at least the electroencephalographic data includes: determining an occurrence of the candidate apnea event in response to the electroencephalographic data satisfying

$$\frac{σ + θ}{α + β} > A$$

where σ, θ, α and β represent an electroencephalographic signal level in a frequency band of 1 Hz to 4 Hz, an electroencephalographic signal level in a frequency band of 4 Hz to 8 Hz, an electroencephalographic signal level in a frequency band of 8 Hz to 13 Hz and an electroencephalographic signal level in a frequency band of 13 Hz to 30 Hz in the electroencephalographic data, respectively, and A represents a preset threshold. In some embodiments, σ, θ, α and β are obtained by performing a spectrum analysis on the electroencephalographic data acquired by the electroencephalographic sensor. In some embodiments, the method further includes: determining a sleep stage of the user according to the electroencephalographic data. In some embodiments, the verifying whether the candidate apnea event is an apnea event or not based on at least the blood oxygen data includes: determining that the candidate apnea event is the apnea event in response to at least one of: a value of the blood oxygen data decreasing by greater than or equal to 5%, the value of the blood oxygen data decreasing by 4% to 5% with a duration of greater than 30 seconds, or the value of the blood oxygen data decreasing by 3% to 4% with a duration of greater than 30 seconds and with awakening.

In some embodiments, the determining a type of the apnea event based on at least the pulse wave data in response to determining that the candidate apnea event is the apnea event includes: determining a first average pulse wave amplitude during the apnea event and a second average pulse wave amplitude prior to the apnea event based on the pulse wave data; and determining the type of the apnea event based on at least the first average pulse wave amplitude and the second average pulse wave amplitude. In some embodiments, the determining the type of the apnea event based on at least the first average pulse wave amplitude and the second average pulse wave amplitude includes: calculating a ratio of the first average pulse wave amplitude to the second average pulse wave amplitude; determining the apnea event as a central apnea event in response to determining that the ratio is less than 0.9; and determining the apnea event as an obstructive apnea event in response to determining that the ratio is within a preset interval [1−ε, 1+ε], where 0<ε≤0.1. In some embodiments, the first average pulse wave amplitude and the second average pulse wave amplitude are determined by identifying characteristic points of the pulse wave data acquired by the photoelectric sensor. In some embodiments, the method further includes: determining a pulse rate of the user according to the pulse wave data. In some embodiments, the determining the type of the apnea event based on at least the first average pulse wave amplitude and the second average pulse wave amplitude includes: determining the type of the apnea event based on at least the first average pulse wave amplitude, the second average pulse wave amplitude and the pulse rate.

In some embodiments, the wearable electronic device is an eye mask electronic device, the electroencephalographic sensor includes an electrode arranged on the eye mask electronic device close to a forehead of the user, and the photoelectric sensor includes a photoelectric device arranged on the eye mask electronic device close to the forehead of the user. In some embodiments, the method further includes: providing data associated with the apnea event and/or the type of the apnea event to outside in a wired or wireless manner.

According to a third aspect of the present disclosure, there is provided a computer-readable storage medium having instructions stored thereon, wherein the instructions, when executed by a processor, cause the processor to perform the method of detecting the apnea according to the second aspect of the present disclosure.

According to a fourth aspect of the present disclosure, there is provided an electronic device, including: an interface module communicatively connected to the electronic device according to the first aspect and configured to receive an information indicating at least one of electroencephalographic data, nasal airflow data, pulse wave data, a candidate apnea event, an apnea event, a type of the apnea event, or a sleep stage; and an output module configured to output the information.

In a case of using the electronic device, the method and/or the computer-readable storage medium described above, a change in the electroencephalographic data and a decrease of the blood oxygen data during sleep apnea may be acquired by the electroencephalographic sensor and used to determine apnea and hypopnea events, and the central apnea and the obstructive apnea may be distinguished according to photoelectric pulse wave characteristics, so as to reduce cost and difficulty of a classification diagnosis. In addition, the solution may be implemented to achieve detection by means of an eye mask, so as to reduce an impact of the device on users' sleep.

### BRIEF DESCRIPTION OF THE DRAWINGS

The preferred embodiments of the present disclosure will be described below in conjunction with the accompanying drawings to make the above and other objectives, features and advantages of the present disclosure clearer.

FIG. 1 shows an application scenario for an exemplary electronic device of detecting apnea according to some embodiments of the present disclosure.

FIG. 2 shows a flowchart of an exemplary method of detecting apnea according to some embodiments of the present disclosure.

FIG. 3 shows a flowchart of an exemplary step of the method shown in FIG. 2.

FIG. 4 shows a flowchart of another exemplary step of the method shown in FIG. 2.

FIG. 5 shows a schematic curve graph of pulse wave amplitude for distinguishing different types of apnea according to some embodiments of the present disclosure.

FIG. 6 shows a schematic arrangement diagram of hardware of the electronic device of detecting the apnea according to some embodiments of the present disclosure.

FIG. 7A and FIG. 7B show exemplary external views of the electronic device of detecting the apnea according to some embodiments of the present disclosure.

FIG. 8 shows a schematic diagram of an electronic device for outputting various information of the electronic device shown in FIG. 6 according to some embodiments of the present disclosure.

### DETAILED DESCRIPTION OF EMBODIMENTS

In order to make the objectives, technical solutions and advantages of the present disclosure clearer, the present disclosure will be further described in detail below in connection with the drawings. It should be noted that the following description is for illustration only and not intended to limit the present disclosure. In the following description, a number of specific details are set forth in order to provide a thorough understanding of the present disclosure. However, it is apparent to those ordinary skilled in the art that these specific details are not necessary to implement the present disclosure. In other examples, in order to avoid confusion with the present disclosure, well-known circuits, materials, or methods are not specifically described.

Throughout the specification, references to “one embodiment,” “an embodiment,” “one example,” or “an example” mean that a specific feature, structure, or characteristic described in conjunction with the embodiment or example is included in at least one embodiment of the present disclosure. Therefore, the phrases “in one embodiment”, “in an embodiment”, “one example” or “an example” appearing in various places throughout the specification do not necessarily refer to the same embodiment or example. Further, specific features, structures or characteristics may be combined in one or more embodiments or examples in any suitable combination and/or sub-combination. In addition, those ordinary skilled in the art should understand that the drawings provided herein are for the illustrative purpose, and the drawings are not necessarily drawn to scale. The term “and/or” as used here includes any and all combinations of one or more related listed items.

In general, the present disclosure relates to a detection of apnea and a classification of apnea. In the present disclosure, whether an apnea event occurs or not during user's sleep is comprehensively determined by using, for example, relevant data (e.g., electroencephalographic data, blood oxygen data, pulse wave data, nasal airflow data, etc.) detected by an electronic device worn by the user (e.g., eye mask, nasal airflow meter, etc.), and the apnea event detected is classified, so as to help the user detect the apnea conveniently, quickly and at low cost without going to the hospital or connecting a large number of devices.

Hereinafter, an electronic device of detecting apnea according to some embodiments of the present disclosure will be described in detail with reference to the drawings.

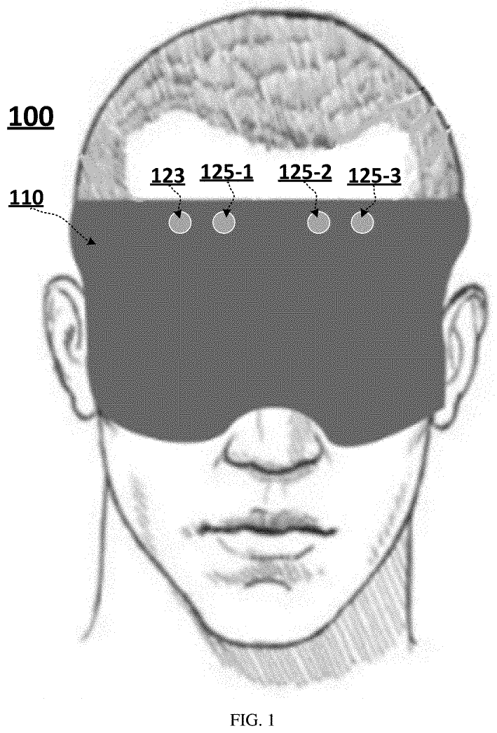

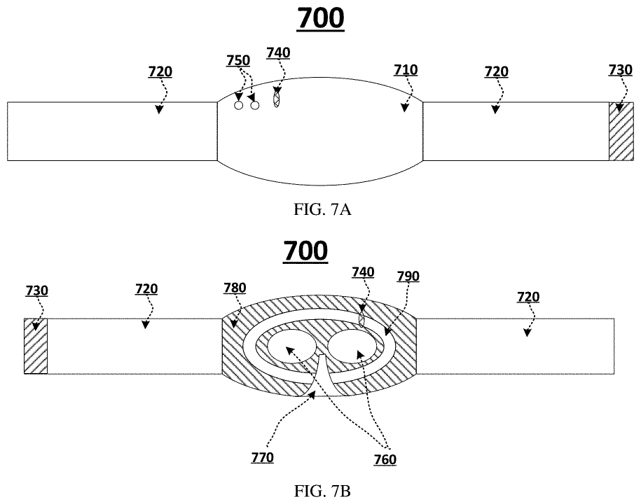

FIG. 1 shows an application scenario for an exemplary electronic device 100 of detecting apnea according to some embodiments of the present disclosure. As shown in FIG. 1, the electronic device 100 may have the form of an eye mask worn by a user. The electronic device 100 may include, for example, an eye mask body 110 and a plurality of sensors provided thereon. The plurality of sensors may include, for example, a photoelectric sensor 123 and electroencephalographic sensors 125-1, 125-2 and 125-3 shown by four dots in FIG. 1. It should be noted that these four dots are only shown for the convenience of understanding. In practice, these four dots are generally arranged inside the eye mask and close to user's skin, and therefore are invisible from the outside (for example, referring to FIG. 7A). In addition, in some embodiments, the dots shown may be part of these sensors, such as electrodes, light sources, photodiodes, photoresistors, etc. used to acquire corresponding data, and the rest of the sensors may be provided at other positions of the eye mask body 710.

In some embodiments, the photoelectric sensor 123 may include a light source to emit infrared light and/or visible light to the user's skin at a position corresponding to the photoelectric sensor 123. According to Lamber-Beer's law, an absorbance of a substance at a wavelength is proportional to a concentration of the substance. Therefore, when light of a constant wavelength is irradiated on human tissue, a measured light intensity after absorption and reflection attenuation by a human tissue may reflect structural characteristics of the tissue of the irradiated part to a certain extent.

Human tissues may be divided into blood tissues and non-blood tissues such as skin, muscles and bones, and the non-blood tissues have a constant light absorption. In blood, a pulsation of venous blood is very weak compared to that of arterial blood and may be ignored. Therefore, it may be considered that a change in light reflected by human tissues is caused only by a filling of arterial blood. Then, under an illumination of a light source with a constant wavelength, a pulse signal of a human body may be indirectly measured by detecting the reflected light intensity.

Similarly, in blood oxygen detection, the light source may be made to emit light with different wavelengths (e.g., infrared light and visible light), and blood oxygen data (e.g., blood oxygen saturation) may be indirectly measured by detecting the intensity of reflected light of different wavelengths because oxygenated hemoglobin (OHb) and hemoglobin (Hb) have different absorption ratios for different wavelengths of light.

In other words, in the embodiment shown in FIG. 1, the photoelectric sensor 123 may measure the pulse wave data and/or the blood oxygen data of the user by detecting the intensity of one or more types of reflected light.

In some embodiments, the electroencephalographic sensors 125-1, 125-2 and 125-3 may include three electrodes, including a reference electrode 125-2 located in the middle and two differential electrodes 125-1 and 125-3 located on both sides. When in use, the required electroencephalographic data may be obtained by separately measuring a potential difference between the differential electrode 125-1 and the reference electrode 125-2 as well as a potential difference between the differential electrode 125-3 and the reference electrode 125-2.

It should be noted that although FIG. 1 shows four circular sensors 123, 125-1, 125-2 and 125-3 arranged side by side, the present disclosure is not limited thereto. In fact, the photoelectric sensor 123 and/or the electroencephalographic sensors 125-1 to 125-3 may be arranged at different positions and/or have different sizes and/or shapes. In addition, the number and/or location of the electroencephalographic sensors are not limited to the specific example shown in FIG. 1. For example, in other embodiments, the photoelectric sensor 123 may be arranged at a position close to a temple of the user inside the eye mask body 110. In other embodiments, the electrodes of the electroencephalographic sensors may be arranged at a plurality of locations (for example, 10 locations, 20 locations, etc.) around the entire head. In addition, in other embodiments where the electronic device 100 is in the form of a helmet-mounted device, the electrodes of the electroencephalographic sensors may be arranged according to 21 electroencephalographic electrode placement points recommended by the International Federation of Clinical Neurophysiology (IFCN), so as to acquire more accurate electroencephalographic data.

In addition, in some embodiments, the electronic device 100 may further include a nasal airflow sensor. The nasal airflow sensor may be used to measure an airflow exhaled by nostrils of the user. For example, the nasal airflow sensor may be, for example, a catheter placed in the nostrils of the user and hung on ears or fixed to the eye mask body 110, which may continuously measure an amount of the airflow exhaled from the nostrils. As described in detail later, it may also be used to detect an apnea event.

In addition, although not shown in FIG. 1, the electronic device 100 may further include other components, such as a controller/processor, a communication module, a power supply module, and so on. Hereinafter, the description may be made in conjunction with FIG. 6 and/or FIG. 7A and FIG. 7B.

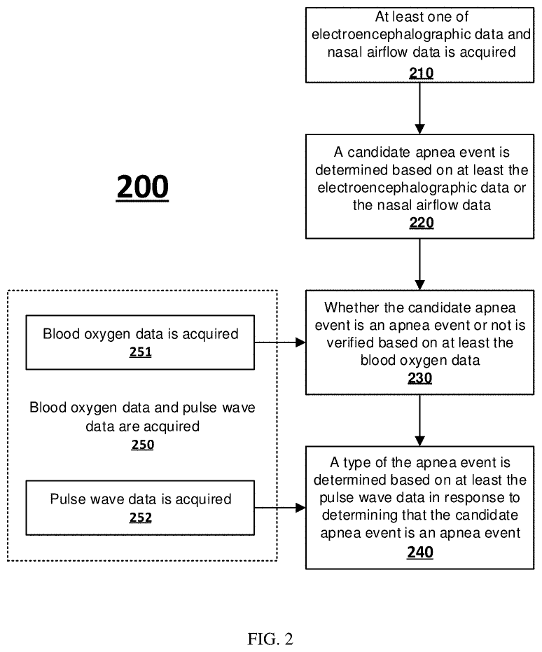

Next, an exemplary method of detecting apnea by using the electronic device 100 shown in FIG. 1 will be described in detail in conjunction with FIG. 2.

FIG. 2 shows a flowchart of an exemplary method 200 of detecting apnea according to some embodiments of the present disclosure. As shown in FIG. 2, the method 200 may include a plurality of steps 210, 220, 230, 240 and 250 (including 251 and 252, for example). However, it should be noted that the method 200 may include fewer steps, more steps, or a step to replace one of the steps, and therefore the embodiments of the present disclosure are not limited thereto. In addition, an order of performing the steps in the method 200 is not limited to the order shown in FIG. 2. For example, step 251 may be performed prior to, subsequent to, or at least partially in parallel to steps 210, 220 and 252. Similarly, step 252 may also be performed prior to, subsequent to, or at least partially in parallel to steps 210, 220 and 251.

The method 200 may begin from step 210. In step 210, at least one of electroencephalographic data and nasal airflow data may be acquired. For example, in the embodiment shown in FIG. 1, the electroencephalographic data of the user may be acquired through the electroencephalographic sensors 125-1 to 125-3. In addition, as described above, the nasal airflow data of the user may also be acquired through the nasal airflow sensor.

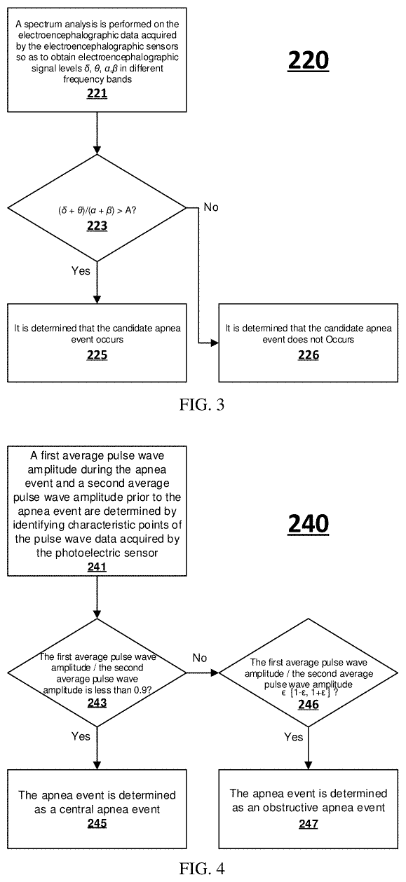

Next, in step 220, a candidate apnea event may be determined based on at least the electroencephalographic data or the nasal airflow data. Specifically, a determination of the candidate apnea event may be described in detail in conjunction with FIG. 3.

FIG. 3 shows a flowchart of the step 220 of the method 200 shown in FIG. 2, in which acquiring the electroencephalographic data by using the electroencephalographic sensor is illustrated by way of example in describing the determination of the candidate apnea event. As shown in FIG. 3, the step 220 may include a plurality of sub-steps 221, 223, 225 and 226. Similar to the above, the step 220 may include fewer sub-steps, more sub-steps, or a step to replace one of the sub-steps, and therefore the embodiments of the present disclosure are not limited thereto. In addition, an order of performing the sub-steps in the step 220 is not limited to the order shown in FIG. 3.

In step 221, a spectrum analysis may be performed on the electroencephalographic data acquired by the electroencephalographic sensors 125-1 to 125-3 so as to obtain electroencephalographic signal levels σ, θ, α and β in different frequency bands, which correspond to an electroencephalographic signal level in a frequency band of 1 Hz to 4 Hz, an electroencephalographic signal level in a frequency band of 4 Hz to 8 Hz, an electroencephalographic signal level in a frequency band of 8 Hz to 13 Hz and an electroencephalographic signal level in a frequency band of 13 Hz to 30 Hz in the electroencephalographic data, respectively. Herein, the “electroencephalographic signal level” in a frequency band refers to an amplitude or intensity of a detected electroencephalographic signal in the frequency band. Therefore, in some embodiments, the spectrum analysis may be performed on the electroencephalographic data to determine the electroencephalographic signal level in each frequency band by detecting the amplitude or intensity of the signal in each frequency band. When a sleep apnea event occurs, α wave activity is generally weakened (i.e., an amplitude or intensity of α wave becomes smaller), and σ wave activity is increased (i.e., an amplitude or intensity of σ wave becomes smaller), and therefore whether a candidate apnea event occurs or not may be determined as follows.

Next, in step 223, whether the candidate apnea event occurs or not may be determined by determining whether Formula (1) is satisfied.

$$\frac{σ + θ}{α + β} > A \qquad Formula ( 1 )$$

where A represents a preset threshold. In some embodiments, A may be an empirical value obtained based on a plurality of tests.

In response to determining that Formula (1) is satisfied, it may be determined in step 225 that the candidate apnea event occurs. Otherwise, it may be determined in step 226 that the candidate apnea event does not occur.

In addition, in some embodiments, a sleep stage of the user may be further determined according to the electroencephalographic data. During sleep, there may be various changes in an electroencephalography, and these changes may vary with a depth of sleep. According to different characteristics of the electroencephalography, sleep may be divided into a non-rapid eye movement sleep and a rapid eye movement sleep, which are distinguished by whether there is a paroxysmal rapid eye movement or not and by different electroencephalographic wave characteristics.

The non-rapid eye movement sleep stage may be divided into four periods according to the electroencephalographic characteristics.
In a hypnagogic period, electroencephalographic waves are dominated by θ waves, without spindle waves or K-complex waves. In fact, it is a transition stage from complete wakefulness to sleep, in which a response to external stimuli is weakened, mental activities enter a floating state, and thinking is divorced from reality.
In a hypohyphnotic period, the electroencephalographic waves include spindle waves and K-complex waves, and a waves are less than 20%. In fact, people have entered a light sleep of a real sleep.
In a moderate sleep period, a waves account for 20% to 50%, and people have entered a moderate sleep.
In a deep sleep period, a waves account for more than 50%, people have entered a deep sleep and is not easy to be awakened.

During the rapid eye movement sleep stage, desynchronized low amplitude electroencephalographic waves with mixed frequency may appear.

Therefore, the sleep stage may be determined according to the electroencephalographic data.

Although FIG. 3 shows an embodiment in which the candidate apnea event is determined based on at least the electroencephalographic data, the specific implementation of step 220 in FIG. 2 is not limited to this. For example, in some embodiments, the candidate apnea event may be determined based on at least the nasal airflow data. Herein, the “nasal airflow data” refers to an amount of the airflow inhaled or exhaled from the nostrils in a unit time, for example, the amount of the airflow inhaled or exhaled every 1 second, 10 seconds, 30 seconds, 1 minute, etc. For example, in some embodiments, when the nasal airflow data indicates that the amount of the nasal airflow per unit time decreases by more than 50%, it may generally be regarded as an occurrence of the candidate apnea event. In addition, in other embodiments, the candidate apnea event may be determined based on a combination of the electroencephalographic data and the nasal airflow data. For example, the occurrence of the candidate apnea event may be determined in response to Formula (1) being satisfied and the amount of the nasal airflow per unit time decreasing by more than 50%

Referring back to FIG. 2, prior to step 230, blood oxygen data (e.g., blood oxygen saturation) may be acquired through the photoelectric sensor 123 in step 251. Then in step 230, whether the candidate apnea event detected in step 220 is an apnea event or not may be verified based on at least the blood oxygen data. In other words, in order to further improve an accuracy of the detection of the apnea event, the verification may be performed from a perspective of the blood oxygen data. However, in other embodiments, the verification step 230 may also be omitted. In some embodiments, in order to verify the apnea event, a Respiratory Disorder Index (RDI) may be used to verify the determination. The Respiratory Disturbance Index may have a determination standard as follows.

In some embodiments, step 230 may be implemented by determining that the candidate apnea event is an apnea event in response to at least one of the followings being satisfied:
a value of the blood oxygen data decreases by greater than or equal to 5%;
the value of the blood oxygen data decreases by 4% to 5% with a duration of greater than 30 seconds; and
the value of the blood oxygen data decreases by 3% to 4% with a duration of greater than 30 seconds and with awakening.

In some embodiments, the awakening may be detected through the sleep stage determined by the electroencephalographic data.

Referring back to FIG. 2, prior to step 240, pulse wave data (e.g., average pulse wave amplitude, pulse rate, etc.) may be acquired through the photoelectric sensor 123 in step 252. Then in step 240, a type of the apnea event may be determined based on at least the pulse wave data in response to determining that the candidate apnea event is an apnea event in step 230. Specifically, a determination of the type of the candidate apnea event may be described in detail in conjunction with FIG. 4.

FIG. 4 shows a flowchart of the exemplary step 240 of the method 200 shown in FIG. 2, in which acquiring the pulse wave data by using the photoelectric sensor 123 is illustrated by way of example in describing the determination of the type of the candidate apnea event. As shown in FIG. 4, the step 240 may include a plurality of sub-steps 241, 243, 245, 246 and 247. Similar to the above, the step 240 may include fewer sub-steps, more sub-steps, or a sub-step to replace one of the sub-steps, and therefore the embodiments of the present disclosure are not limited thereto. In addition, an order of performing the sub-steps in the step 240 is not limited to the order shown in FIG. 4.

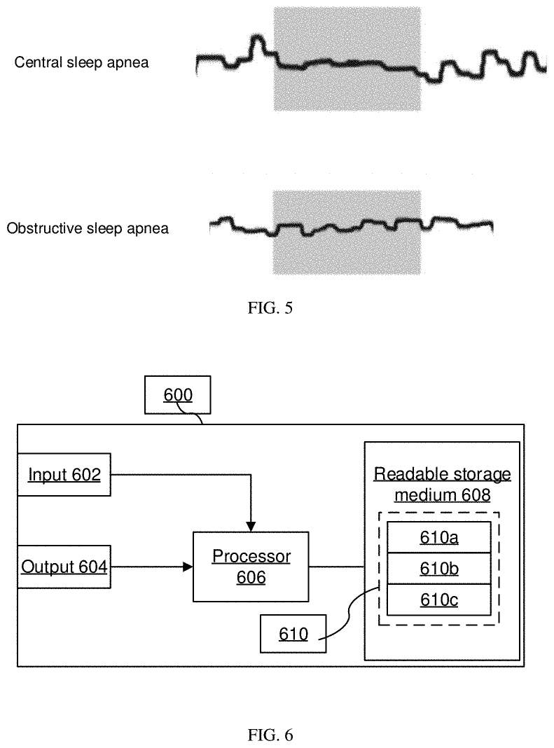

In general, the pulse wave amplitude during an obstructive apnea event does not change significantly compared to that prior to the event and subsequent to the event, while the pulse wave amplitude during a central apnea event may change significantly compared to that prior to the event and subsequent to the event. A reason for this phenomenon is that the obstructive apnea event is resulted by apnea or hypoventilation caused by an obstruction of the respiratory tract due to a collapse of the upper respiratory tract (for example, due to a hypertrophic root of the tongue becoming loose during sleep), which has no obvious effect on the ratio of pulse wave amplitude described above. In contrast, the central apnea event is caused by a failure of a nerve conduction that controls an operation of the respiratory system, in which the respiratory tract is unblocked but the respiratory muscles do not operate. Since the nerves that control the respiratory system also affect a heartbeat, and the pulse wave amplitude is significantly affected by the heartbeat, the pulse wave amplitude during the event may change significantly compared to that prior to and subsequent to the event. This point is more intuitively illustrated in FIG. 5.

FIG. 5 shows a schematic curve graph of pulse wave amplitude for distinguishing different types of apnea according to some embodiments of the present disclosure. As shown in FIG. 5, in a curve corresponding to the central apnea event, the average pulse wave amplitude during the event is lower than that prior to and subsequent to the event, while in a curve corresponding to the obstructive apnea event, the average pulse wave amplitude during the event does not change significantly compared to that prior to and subsequent to the event. Therefore, the type of the apnea event may be distinguished based on the average pulse wave amplitude.

For example, in sub-step 241, a first average pulse wave amplitude during the apnea event and a second average pulse wave amplitude prior to the apnea event may be determined by identifying characteristic points of the pulse wave data acquired by the photoelectric sensor 123. In other embodiments, the second average pulse wave amplitude may also be an average pulse wave amplitude subsequent to the apnea event, or an average pulse wave amplitude prior to and subsequent to the apnea event. However, considering an end of the apnea event may not be determined clearly, the average pulse wave amplitude prior to the apnea event may be selected as the second average pulse wave amplitude. For example, the characteristic points of the pulse wave shown in FIG. 5 may be identified, so as to determine, for example, one or more of a starting point, a main wave point, a dicrotic notch point, a dicrotic wave and so on. For example, in some embodiments, the starting point and the main wave point may be determined, then the pulse wave amplitude and/or the pulse rate may be determined, and then the average pulse wave amplitude prior to and subsequent to the event and that during the event may be determined.

Specifically, in some embodiments, the characteristic points of the pulse wave may be identified by using, for example, a differential threshold method. This method is a simple and efficient method to identify peaks and troughs of periodic signals. In a classification of respiratory events, pulse peaks and pulse troughs may be the main characteristic points involved. The specific method may include obtaining a differentiation by point difference of the pulse wave data, and obtaining the peak and trough of the pulse wave according to differential zero crossing. The point with a differential from positive to negative is a peak point, and the point with a differential from negative to positive is a trough point. In addition, in order to improve an efficiency of recognition, an adaptive threshold may be introduced, and the threshold may be updated according to a specific cycle. Within a specific threshold range, characteristic points may be detected and an amount of calculations may be reduced.

Next, in step 243, when it is determined that a ratio of the first average pulse wave amplitude to the second average pulse wave amplitude is less than 0.9, the apnea event may be determined as a central apnea event in step 245. In addition, when it is determined in step 246 that the ratio is within a preset interval [1−ε, 1+ε], the apnea event may be determined as an obstructive apnea event in step 247, where 0<ε≤0.1. In addition, in other embodiments, step 243 and step 246 may be performed in a different order. For example, step 243 may be performed prior to, subsequent to, or at least partially in parallel to step 246. Similarly, step 245 and step 247 may be performed in a different order. For example, step 245 may be performed prior to, subsequent to, or at least partially in parallel to step 247.

Therefore, the apnea event may be detected and classified by using the method 200 shown in FIG. 2, FIG. 3 and FIG. 4, the change in the electroencephalographic data and the decrease in the blood oxygen during the sleep apnea may be acquired by the electroencephalographic sensors and used to determine apnea and hypopnea events, and the central apnea event and the obstructive apnea may be distinguished according to the characteristics of photoelectric pulse wave, so as to reduce the cost and difficulty of the classification diagnosis. In addition, the method may be implemented to achieve detection relying on an eye mask, so as to reduce the impact of the device on the user's sleep.

In addition, in some embodiments, the method 200 may further include determining the pulse rate of the user according to the pulse wave data. In some embodiments, step 240 may further include determining the type of the apnea event based on at least the first average pulse wave amplitude, the second average pulse wave amplitude, and the pulse rate.

In some embodiments, the pulse rate may have an auxiliary function in determining the type of the apnea event. For example, as described above, the obstructive apnea event is caused by the obstruction of the respiratory tract. After a short period of obstructive apnea, the pulse rate generally does not change significantly. In contrast, the central apnea event is caused by a failure of a nerve conduction that controls the operation of the respiratory system, in which the respiratory tract is unblocked but the respiratory muscles do not operate. Therefore, the nerves that control the respiratory system also affect the heartbeat. When the central apnea event occurs, the pulse rate may change significantly. Therefore, after determining that the apnea event occurs, the pulse rate during the event may be compared to that prior to and subsequent to the event to assist in the classification of the apnea event. For example, in some embodiments, when the pulse rate during the apnea event changes by more than 10% compared to that prior to and subsequent to the apnea event, it may be considered that the pulse rate during the apnea event changes significantly compared to that prior to and subsequent to the apnea event. Then the type of the event may be finally determined combined with the change in the (average) pulse amplitude.

In addition, in some embodiments, the electronic device 100 may be an eye mask electronic device as shown in FIG. 1 or FIG. 7A, FIG. 7B. The electroencephalographic sensors 125-1 to 125-3 may include electrodes arranged on the eye mask electronic device close to a forehead of the user. The photoelectric sensor 123 may include a photoelectric device (e.g., a visible light source, an infrared light source, a photodiode, a photoresistor, etc.) arranged on the eye mask electronic device close to the forehead of the user.

In addition, in some embodiments, the electronic device 100 may further include a communication module configured to provide data associated with the apnea event and/or the type of the apnea event to outside in a wired or wireless manner.

FIG. 6 shows a schematic arrangement diagram of hardware of an electronic device 600 (for example, the electronic device 100 shown in FIG. 1, the eye mask shown in FIG. 7A and FIG. 7B) of detecting apnea according to some embodiments of the present disclosure. The hardware arrangement 600 may include a processor or controller 606 (e.g., a digital signal processor (DSP), a central processing unit (CPU), etc.). The processor 606 may be a single processing unit or a plurality of processing units for executing different actions of the processes described herein. The arrangement 600 may also include an input unit 602 for receiving signals from other entities, and an output unit 604 for providing signals to other entities. The input unit 602 and the output unit 604 may be arranged as a single entity or separate entities.

In addition, the arrangement 600 may include at least one readable storage medium 608 in the form of a non-volatile or volatile memory, such as an electrically erasable programmable read-only memory (EEPROM), a flash memory, and/or a hard drive. The readable storage medium 608 may contain a computer program 610 that includes code/computer readable instructions, which when executed by the processor 606 in the arrangement 600 cause the hardware arrangement 600 and/or an electronic device including the hardware arrangement 600 to perform, for example, the flow described above in conjunction with FIG. 2 to FIG. 4 and any modification thereof.

The computer program 610 may be configured as a computer program code having an architecture of, for example, computer program modules 610A to 610C. Therefore, in an exemplary embodiment where the hardware arrangement 600 is used in, for example, the electronic device 100, the eye mask 700, or another electronic device, the code in the computer program of the arrangement 600 may include: a module 610A for determining the candidate apnea events based on at least the electroencephalographic data or the nasal airflow data. Furthermore, in some embodiments, the code in the computer program of the arrangement 600 may further include a module 610B for verifying whether the candidate apnea event is the apnea event or not based on at least the blood oxygen data. In addition, in some embodiments, the code in the computer program of arrangement 600 may further include a module 610C for determining the type of the apnea event based on at least the pulse wave data in response to determining that the candidate apnea event is the apnea event.

The computer program module may essentially execute various actions in the flow shown in FIG. 2 to FIG. 4 so as to simulate the electronic device 100, the eye mask 700 or the other electronic device. In other words, different computer program modules may correspond to different units or modules in the electronic device 100, the eye mask 700 or the other electronic device when being executed in the processor or controller 606.

Although the code means in the embodiment disclosed above in conjunction with FIG. 6 is implemented as a computer program module, which when executed in the processor 606 causes the hardware arrangement 600 to execute the actions described above in conjunction with FIG. 2 to FIG. 4, in alternative embodiments, at least one of the code means may be at least partially implemented as a hardware circuit.

The processor may be a single CPU (Central Processing Unit), but may also include two or more processing units. For example, the processor may include a general-purpose microprocessor, an instruction set processor and/or a related chipset and/or a special-purpose microprocessor (for example, an application specific integrated circuit (ASIC)). The processor may further include on-board memory for caching purposes. The computer program may be carried by a computer program product connected to the processor. The computer program product may include a computer readable medium having a computer program stored thereon. For example, the computer program product may be a flash memory, a random access memory (RAM), a read-only memory (ROM), an EEPROM, and the above-mentioned computer program modules may be distributed to different program products in the form of memory in the electronic device in an alternative embodiment.

FIG. 7A and FIG. 7B show exemplary external views of the electronic device of detecting apnea or the eye mask 700 according to some embodiments of the present disclosure. An outer side view of the eye mask 700 is shown in FIG. 7A, and an inner side view of the eye mask 700 is shown in FIG. 7B. In addition, in the embodiment shown in FIG. 7A and FIG. 7B, the eye mask 700 may further include various components that are invisible from the outside, such as the various components shown in FIG. 6.

As shown in FIG. 7A, in some embodiments, the eye mask 700 may include a main body 710 and two second body parts 720 located on both sides of the main body 710, respectively. In some embodiments, the eye mask 700 may be an integrated electronic device, that is, all sensors, controllers and other related devices are provided on the eye mask 700, and then the eye mask 700 may operate independently. In other embodiments, the eye mask 700 may be a split-type electronic device. For example, the sensors for sensing the electroencephalographic data, the pulse wave data, the blood oxygen data, etc., may be provided on the eye mask 700, and the controller for processing the data, the communicator for transmitting data and/or instructions, etc. may be arranged in an external device, and they may be communicatively coupled via a wired line or a wireless line.

For example, as shown in FIG. 7A, the eye mask 700 may further include a conductive fixing member 750. In some embodiments, circuits such as the external controller and communicator may be communicatively connected to the various sensors provided on the eye mask 700 through the conductive fixing member 750. In some embodiments, the conductive fixing member 750 may be, for example, a magnetic buckle. In some embodiments, the magnetic buckle may include a female magnetic buckle 750 arranged on the main body 710 of the eye mask 700 and a male magnetic buckle arranged on the external controller (for example, the electronic device 600 shown in FIG. 6). The female magnetic buckle 750 may be electrically connected to the sensors and a heating part 790 through wires inside the main body 710.

In addition, as shown in FIG. 7A, the eye mask 700 may further include an opening 740. In some embodiments, at least a part of the external device (for example, the electronic device 600 shown in FIG. 6) may pass through the opening 740 to contact forehead skin of the user wearing the eye mask 700, so as to acquire, for example, the electroencephalographic data and the pulse wave data. In addition, in other embodiments, the eye mask 700 may further include a plurality of sensors arranged inside the eye mask 700 and a communication interface (for example, a USB Type C interface) communicatively coupled with these sensors. In this case, the eye mask 700 may not be provided with the opening 740, but allows the external device (for example, the electronic device 600 shown in FIG. 6) to communicate with the sensors through the communication interface, so as to acquire the corresponding data.

In some embodiments, one or more of the opening 740, the controller (for example, the processor 606 shown in FIG. 6), a light shielding layer 780, and the heating part 790 may be provided on the main body 710.

In some embodiments, one end of each second body part 720 is connected to the main body 710, and when the eye mask 700 surrounds the wearer's head (i.e., when the wearer uses the eye mask), the other ends of both second body parts 720 are connected by an adhesive part 730 (for example, a velcro). The main body 710 and the second body parts 720 may have a relatively large width to distribute a pressure applied by the eye mask 700 to different positions of the wearer's head, so as to avoid causing discomfort to the wearer due to excessive local pressure.

As shown in FIG. 7B, the light shielding layer 780 may be provided on the inner side of the eye mask main body 710. The light shielding layer 780 is arranged such that when the wearer uses the eye mask 700, (for example, two) first recesses 760 are provided in a portion of the light shielding layer 780 overlapping an eye area of the wearer. In some embodiments, the light shielding layer 780 may fit the wearer's face, so as to completely shield light. Since the first recesses 760 are provided in the light shielding layer 780, the light shielding layer 780 may not press eyeballs of the wearer during use.

In addition, in some embodiments, when the width of the main body 710 of the eye mask 700 (that is, a size in a top-to-bottom direction in FIG. 7B) is relatively large, a second recess 770 may be further provided in the light shielding layer 780, so that the light shielding layer 780 may fit the wearer's nose to prevent light leakage when the wearer uses the eye mask. In some embodiments, the opening 740 may be arranged such that when the wearer uses the eye mask, the opening 740 may be located outside the area of the eye mask main body 710 overlapping the wearer's eye area so as to prevent light leakage. In addition, in some embodiments, the eye mask 700 may further include a heating part 790 arranged inside the light shielding layer 780. In some embodiments, the heating part 790 may contain graphene.

In some embodiments, the controller may further include a power interface. When the wearer uses the eye mask, the power interface may be connected to a power source (for example, a battery or an AC power source) to supply power to the eye mask.

In a case of using the eye mask 700, the change in the electroencephalographic data and the decrease of the blood oxygen during the sleep apnea may be acquired by the electroencephalographic sensor and used to determine apnea and hypopnea events, and the central apnea event and the obstructive apnea event may be distinguished according to characteristics of the photoelectric pulse wave, so as to reduce cost and difficulty of the classification diagnosis. In addition, because the one-piece eye mask is light and easy to use, an adverse effect on the user's sleep may be reduced, so that the apnea event may be detected more accurately.

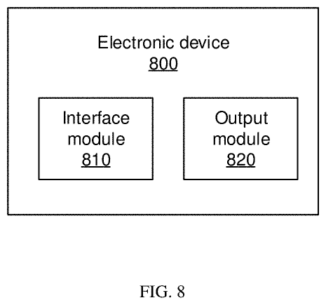

FIG. 8 shows a schematic diagram of an electronic device 800 for outputting various information of the electronic device shown in FIG. 6 according to some embodiments of the present disclosure. As shown in FIG. 8, the electronic device 800 may include an interface module 810 and an output module 820. In some embodiments, the interface module 810 may be communicatively connected to the electronic device of detecting the apnea described above (for example, the electronic device 600) and configured to receive information indicating at least one of electroencephalographic data, nasal airflow data, pulse wave data, a candidate apnea event, an apnea event, a type of the apnea event, or a sleep stage. In some embodiments, the output module 820 may be configured to output at least a portion of the information received by the interface module 810.

For example, in a case that the electronic device 800 and the electronic device 600 are the same device, the interface module 810 may be, for example, a bus structure of the electronic device, and the output module 820 may be, for example, an output module such as a display or a speaker of the electronic device. For another example, in a case that the electronic device 800 and the electronic device 600 are different devices, the interface module 810 may be, for example, a communication module of the electronic device 800 (for example, wired communication module such as a USB port, a chip, an Ethernet interface and a chip, or a wireless communication module such as Bluetooth, Wi-Fi, NFC, etc.), and the output module 820 may be, for example, an output module such as a display or a speaker of the electronic device 800.

So far, the present disclosure has been described in conjunction with the preferred embodiments. It should be understood that those skilled in the art may make various other changes, substitutions and additions without departing from the spirit and scope of the present disclosure. Therefore, the scope of the present disclosure is not limited to the specific embodiments described above, but should be defined by the appended claims.

What is claimed is:

1. An electronic device for detecting apnea, comprising:
at least one of an electroencephalographic sensor configured to detect electroencephalographic data of a user or a nasal airflow sensor configured to detect nasal airflow data of the user; and
a controller communicatively coupled to the at least one of the electroencephalographic sensor or the nasal airflow sensor, wherein the controller is configured to determine a candidate apnea event based on at least the electroencephalographic data or the nasal airflow data.

2. The electronic device of claim 1, further comprising:
a photoelectric sensor communicatively coupled to the controller and configured to detect blood oxygen data of the user, wherein the controller is further configured to verify whether the candidate apnea event is an apnea event or not based on at least the blood oxygen data.

3. The electronic device of claim 2, wherein the photoelectric sensor is further configured to detect pulse wave data of the user; and
wherein the controller is further configured to determine a type of the apnea event based on at least the pulse wave data in response to determining that the candidate apnea event is the apnea event.

4. The electronic device of claim 1, wherein the controller is configured to determine the candidate apnea event based on at least the electroencephalographic data by:
determining an occurrence of the candidate apnea event in response to the electroencephalographic data satisfying
$$\frac{σ + θ}{α + β} > A$$
where σ, θ, α and β represent an electroencephalographic signal level in a frequency band of 1 Hz to 4 Hz, an electroencephalographic signal level in a frequency band of 4 Hz to 8 Hz, an electroencephalographic signal level in a frequency band of 8 Hz to 13 Hz and an electroencephalographic signal level in a frequency band of 13 Hz to 30 Hz in the electroencephalographic data, respectively, and A represents a preset threshold.

5. The electronic device of claim 4, wherein σ, θ, α and β are obtained by performing a spectrum analysis on the electroencephalographic data acquired by the electroencephalographic sensor.

6. The electronic device of claim 4, wherein the controller is further configured to determine a sleep stage of the user according to the electroencephalographic data.

7. The electronic device of claim 1, wherein the controller is configured to determine the candidate apnea event based on at least the nasal airflow data by:
determining an occurrence of the candidate apnea event in response to the nasal airflow data indicating that an airflow per unit time of the nasal airflow decreases by more than 50%.

8. The electronic device of claim 2, wherein the controller is configured to verify whether the candidate apnea event is an apnea event or not based on at least the blood oxygen data by:
determining that the candidate apnea event is the apnea event in response to at least one of: a value of the blood oxygen data decreasing by greater than or equal to 5%, the value of the blood oxygen data decreasing by 4% to 5% with a duration of greater than 30 seconds, or the value of the blood oxygen data decreasing by 3% to 4% with a duration of greater than 30 seconds and with awakening.

9. The electronic device of claim 3, wherein the controller is configured to determine a type of the apnea event based on at least the pulse wave data in response to determining that the candidate apnea event is the apnea event by:
determining a first average pulse wave amplitude during the apnea event and a second average pulse wave amplitude prior to the apnea event based on the pulse wave data; and
determining the type of the apnea event based on at least the first average pulse wave amplitude and the second average pulse wave amplitude.

10. The electronic device of claim 9, wherein the controller is configured to determine the type of the apnea event based on at least the first average pulse wave amplitude and the second average pulse wave amplitude by:
calculating a ratio of the first average pulse wave amplitude to the second average pulse wave amplitude;
determining the apnea event as a central apnea event in response to determining that the ratio is less than 0.9; and
determining the apnea event as an obstructive apnea event in response to determining that the ratio is within a preset interval [1−ε, 1+ε], where 0<ε≤0.1.

11. The electronic device of claim 9, wherein the first average pulse wave amplitude and the second average pulse wave amplitude are determined by identifying characteristic points of the pulse wave data acquired by the photoelectric sensor.

12. The electronic device of claim 9, wherein the controller is further configured to determine a pulse rate of the user according to the pulse wave data.

13. The electronic device of claim 12, wherein the controller is configured to determine the type of the apnea event based on at least the first average pulse wave amplitude and the second average pulse wave amplitude by:
determining the type of the apnea event based on at least the first average pulse wave amplitude, the second average pulse wave amplitude and the pulse rate.

14. The electronic device of claim 2, wherein the electronic device is an eye mask electronic device, the electronic device includes the electroencephalographic sensor, the electroencephalographic sensor comprises an electrode arranged on the eye mask electronic device close to a forehead of the user, and the photoelectric sensor comprises a photoelectric device arranged on the eye mask electronic device close to the forehead of the user.

15. The electronic device of claim 1, further comprising:
a communication module configured to provide data associated with the apnea event and/or the type of the apnea event to outside devices in a wired or wireless manner.

16. A computer-readable storage medium having instructions stored thereon, wherein the instructions, when executed by a processor, cause the processor to:
acquire blood oxygen data and pulse wave data;
acquire at least one of electroencephalographic data or nasal airflow data;
determine a candidate apnea event based on at least the electroencephalographic data or the nasal airflow data;
verify whether the candidate apnea event is an apnea event or not based on at least the blood oxygen data; and
determine a type of the apnea event based on at least the pulse wave data in response to determining that the candidate apnea event is the apnea event.

17. An electronic output device, comprising:
an interface module communicatively connected to the electronic device of claim 1, and configured to receive information indicating at least one of electroencephalographic data, nasal airflow data, pulse wave data, a candidate apnea event, an apnea event, a type of the apnea event, or a sleep stage; and
an output module configured to output the information.
